(19) United States
(12) Patent Application Publication
(10) Pub. No.: US 2023/0210696 A1
(43) Pub. Date: Jul. 6, 2023

(54) EYE PATCH DEVICE

(71) Applicant: FocalPoint Surgical Innovations LLC, Colts Neck, NJ (US)

(72) Inventor: Anish Kapur, Colts Neck, NJ (US)

(21) Appl. No.: 18/093,988

(22) Filed: Jan. 6, 2023

Related U.S. Application Data
(60) Provisional application No. 63/297,059, filed on Jan. 6, 2022.

(51) Int. Cl. A61F 9/04

(52) U.S. Cl. CPC A61F 9/04

(57) ABSTRACT

A reversible eye patch configured to be worn on either the left or right eye of the patient, including a headband having a longitudinal axis and a mount assembly coupled to a portion of an exterior surface of the headband that defines a circumferentially extending pocket that further defines an elliptically shaped central opening in the mount assembly. A major axis of the elliptically shaped central opening is positioned at an acute angle β with respect to the longitudinal axis of the headband. The eye patch also includes an eye shield having a generally ovoid shape that is configured to be selectably seated within the pocket of the mount assembly. A strap extends outwardly from a portion of the mount assembly along an axis generally coaxial to the major axis of the elliptically shaped central opening and has a distal end portion that is configured to be selectively and releasably coupled to a portion of the headband so that the eye shield can be secured about the selected eye of the patient.

### CROSS-REFERENCE TO RELATED APPLICATION

The present application claims priority to U.S. Provisional Patent Application No. 63/297,059, filed Jan. 6, 2022, titled “EYE PATCH DEVICE,” the disclosure of which is incorporated herein by reference in its entirety.

### FIELD OF THE INVENTION

The present disclosure generally relates to eye patch devices that are improved in appearance, eye mounting relationship and medical adaptability in its construction and utilization. In particular, it relates to a disposable eye patch device that is configured to selectively cover one eye of a patient.

### BACKGROUND

Eye patches are known in the art. For example, U.S. Pat. No. 4,682,371 discloses a protective eye patch. The '371 patch has several tabs for securing the patch to a patient's eye. U.S. Pat. No. 3,068,863 discloses a patch designed to keep the eye closed. U.S. Pat. No. 3,092,103 discloses a patch with a cushion material at the edge that allows the patient's eye to move underneath the eye patch. U.S. Pat. No. 3,908,645 for an ophthalmic therapeutic pressure bandage with a conformable, permeable carrier tape.

Conventional disposable eye patches typically have one or more adhesive strips that are configured to secure the eye patch to the patient's face. However, a difficulty with this arrangement is that the adhesive strips of the eye patch may cause skin and/or eye irritation, which increases the likelihood that the patient may remove the patch or undesirably touch or interact with the eye patch. For example, the adhesive of the eye patch may cause irritation of the patient's skin or eyebrow, and the proximity of the eye patch near the eye may also cause irritation. Furthermore, because the eye patch is disposable, the eye patch may need to be replaced on a daily basis or even more frequently, making use of the eye patch fairly expensive.

An alternative to the disposable adhesive eye patch is an eye patch that is attached to a portion of the patient's eyeglasses, such as the eye patch described in U.S. Pat. No. 5,927,279. In this art, an eye patch is disclosed that is formed from a fabric body that is attached at one end to a nose pad of the eyeglass frames. The other end of the fabric body includes slots that receive an arm of the eyeglass frames. A difficulty with this eye patch is that the fabric body of the eye patch may contact and irritate the eye. Furthermore, repeatedly attaching the eye patch to the nose pad and removing the eye patch from the nose pad increases the likelihood that the nose pad may be broken, especially when performed by a child. Also, this eye patch must be used with eyeglass frames that have a particular structure that can be adapted to accept the eye patch.

As disclosed, the eye patch provides a lightweight, easily mounted and removed eye socket cover device that assures a precise and trim coverage and fit about an eye socket area, and that is primarily for medical usage where, for example, an eye has been damaged, operated on or is to be protected. It eliminates difficulties which have been heretofore encountered in eye patch devices and their utilization. As shown, the eye patch is configured to provide full protection without unsightliness and to substantially correspond in shape to an eyeglass while, at the same time, enabling a fully protective covering of the eye, to either prevent or permit normal eyelash movement as desired, and to provide a securely mounted positioning on the surrounding bone structure of an eye socket, and without unsightly ties or projecting tabs. As constructed, it enables a normal lid winking relation, if desired, and also if the eyelashes are extremely long and would normally be hindered in their movement.

Thus, there is a continuing need for an eye patch addressing one or more of the problems that are stated above.

### SUMMARY

The present system provides, among other things, an eye patch configured to provide full protection while allowing for comfortable positioning of the eye patch about the patient's head, which allows for the secure positioning of an eye shield in substantially correspondence to the contours of the surrounding bone structure of the patient's eye socket without the necessity for adhesively securing the eye patch to the patient's skin.

In one embodiment, the eye patch can include a headband, a mount assembly, an eye shield, and a strap. In this aspect, at least a portion of the headband is formed of elastic material to allow for a comfortable compressive fit of the headband about patient head that vary in size. In a further aspect, the mount assembly has first and second sheets of material that are positioned in opposition and are connected to each other about their respective circumferential edges. As shown, connected first and second sheets define an elliptically shaped central opening in the mount assembly and a circumferentially extending pocket. Further, in a non-worn position, the mount assembly is coupled to a portion of an exterior surface of the headband such that a major axis of the elliptically shaped central opening is positioned at an acute angle β with respect to a longitudinal axis of the headband.

Further, the eye shied has a generally ovoid shape and a peripheral edge and is configured to be selectably seated within the pocket of the mount assembly. Finally, an at least partially elastic strap is mounted to and extends outwardly from a portion of the circumferential edges of the first and second sheets of the mount assembly along an axis generally coaxial to the major axis of the elliptically shaped central opening. As shown, a distal end portion of the strap is configured to be selectively and releasably coupled to a portion of the headband to assure the seated secured relationship of the eye shield positioned therein the mount assembly to the eye socket bone construction is achieved.

Various implementations described in the present disclosure can include additional systems, methods, features, and advantages, which can not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### DESCRIPTION OF THE FIGURES

The features and components of the following figures are illustrated to emphasize the general principles of the present disclosure. Corresponding features and components throughout the figures can be designated by matching reference characters for the sake of consistency and clarity.

FIG. 1 shows an eye patch constructed in accordance with the invention in a normal bridging mounted relation over an eye socket of a wearer to give full protection to and cover his left eye.

FIG. 2 shows an eye patch constructed in accordance with the invention in a normal bridging mounted relation over an eye socket of a wearer to give full protection to and cover his right eye, in which the eye patch has the same construction as the eye patch shown in FIG. 1.

FIG. 3 is a front plan view of an eye patch in a non-worn position, showing adhesive tabs for removably securing the device in a wearing position on a wearers left or right eye and showing an eye shield mounted therein the eye patch.

FIG. 4 is an enlarged view of the eye patch of FIG. 3, showing an eye shield removably disposed within an elliptically shaped central opening defined in the mount assembly of the eye patch, and showing the semi major axis of the elliptically shaped central opening being positioned at an acute angle γ with respect to the longitudinal axis of the headband.

FIG. 5 is a back plan view of an eye patch in a non-worn position, showing adhesive tabs for removably securing the device in a wearing position on a wearer's left or right eye and showing an eye shield mounted therein the eye patch.

FIG. 6 shows a front side elevational view of the eye patch of FIG. 1, in a mounted position, in which the eye patch is secured to the wearer's head and is positioned in a normal bridging mounted relation over an eye socket of a wearer to give full protection to and cover his left eye.

FIG. 7 shows a right-side elevational view of the eye patch of FIG. 1, in a mounted position, in which the eye patch is secured to the wearer's head and is positioned in a normal bridging mounted relation over an eye socket of a wearer to give full protection to and cover his left eye.

FIG. 8 shows a left-side elevational view of the eye patch of FIG. 1, in a mounted position, in which the eye patch is secured to the wearer's head and is positioned in a normal bridging mounted relation over an eye socket of a wearer to give full protection to and cover his left eye.

FIG. 9 shows a back side elevational view of the eye patch of FIG. 1, in a mounted position, in which the eye patch is secured to the wearer's head and is positioned in a normal bridging mounted relation over an eye socket of a wearer to give full protection to and cover his left eye.

FIG. 10 is a perspective view of the eye patch, showing an eye shield being selective received through a selectively sealable pocket opening in a mount assembly of the eye patch so that the eye shield can be removably disposed within a central opening defined in the mount assembly of the eye patch so that the inner surface of the eye shield is spaced from the wearer's eye.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “an aperture” can include two or more such apertures unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment.

Disclosed are components that can be used to perform the disclosed methods and systems. These and other components are disclosed herein, and it is understood that when combinations, subsets, interactions, groups, etc. of these components are disclosed that while specific reference to each various individual and collective combinations and permutation of these cannot be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems can be understood more readily by reference to the following detailed description of preferred embodiments and the examples included therein and to the Figures and their previous and following description.

The present system provides, among other things, provides an eye patch system and method that is configured to protect the eye from blunt trauma and advantageously provides a maximum degree of comfort, while being easily attached and removed from the face of the wearer without causing pain, further injury, or improper contact or pressure being applied to the eye.

As disclosed, the eye patch 10 provides a lightweight, easily mounted and removed eye patch 10 that assures a precise and trim coverage and fit of an eye shield 20 about an eye socket area, and that is primarily for medical usage where, for example, an eye has been damaged, operated on or is to be protected. The eye patch eliminates difficulties which have been heretofore encountered in eye patch devices and their utilization. As shown, the eye patch is configured to provide full protection while allowing for comfortable positioning of the eye patch about the patient's head, which allows for the secure positioning of an eye shield in substantially correspondence to the contours of the surrounding bone structure of the patient's eye socket without the necessity for adhesively securing the eye patch to the patient's skin. As constructed, the eye patch enables a normal lid winking relation, if desired, and also if the eyelashes are extremely long and would normally be hindered in their movement. As shown, it is contemplated that the disclosed eye patch is reversible and, as such, can be configured to be worn on either the left or right eye of the patient as desired.

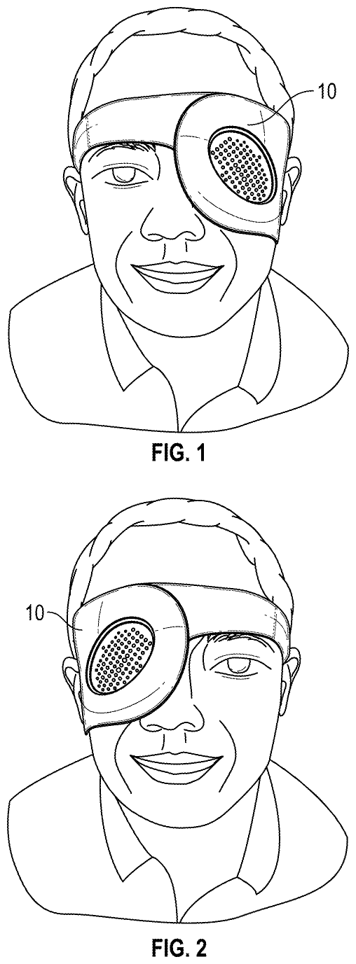

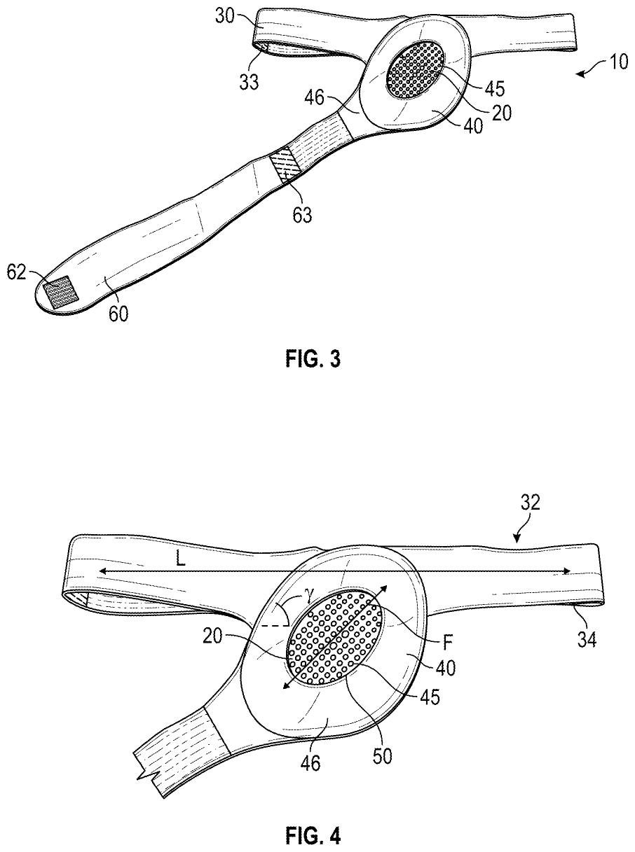

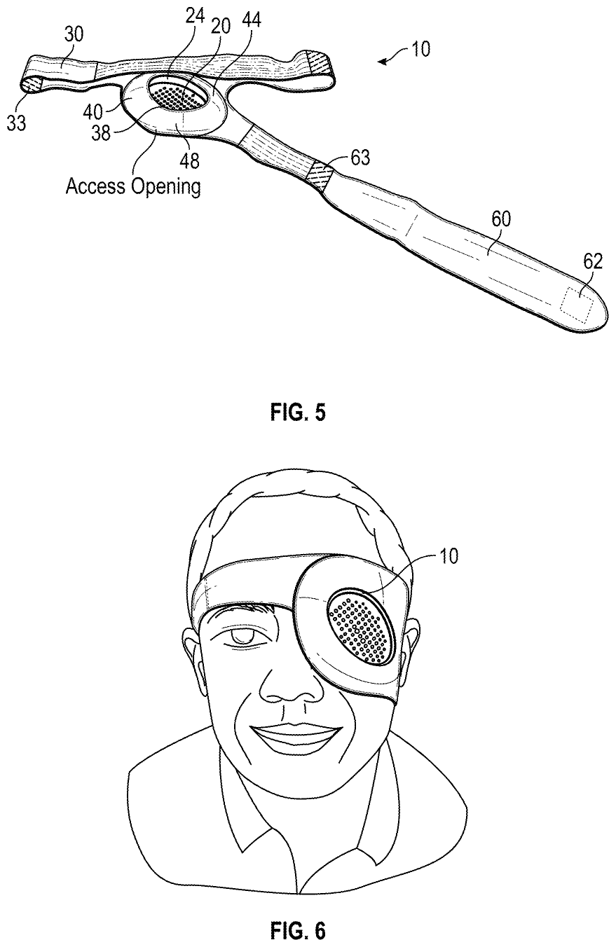

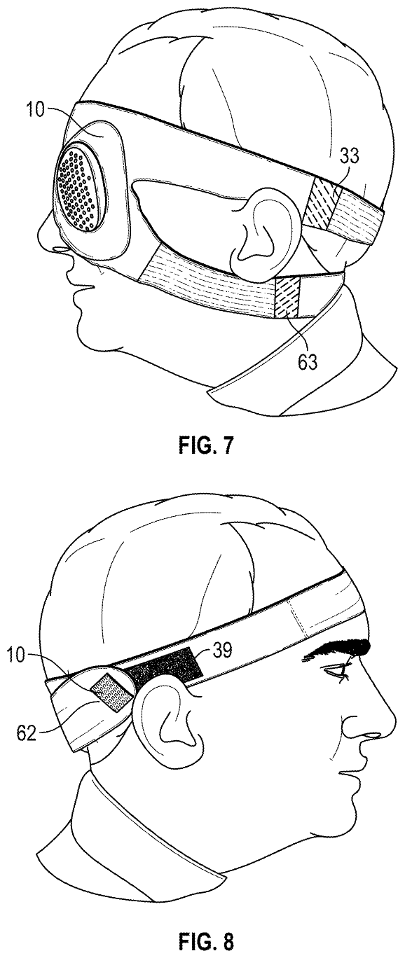

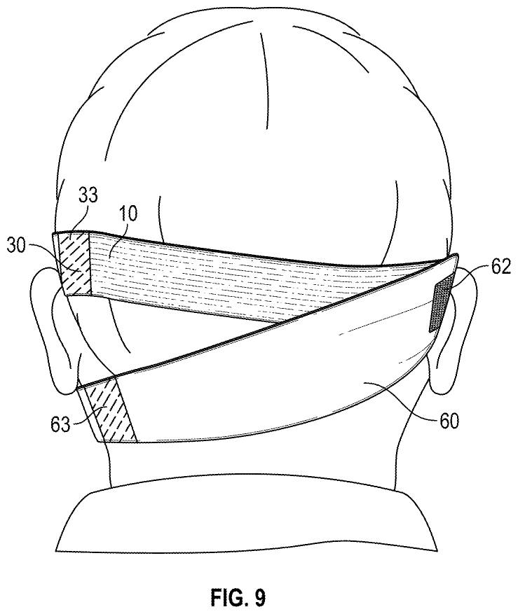

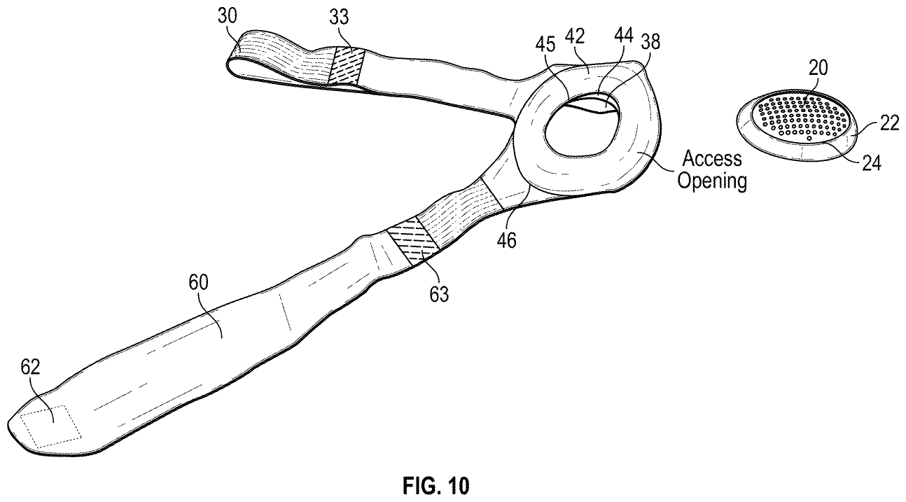

As illustrated in FIGS. 1-4, the eye patch 10 can comprise a headband 30, a mount assembly 40, and a strap 60. The headband can form a continuous loop or it can be discontinuous, in a non-worn position, but can be positioned in a desired loop configuration, in a worn mounted position, about the patient's head. As one skilled in the art will appreciate, the exact dimensions of the headband can be varied to fit persons of various sizes, and to take in consideration of the physical characteristics of the materials selected for the headband. As illustrated, the headband 30 has a longitudinal axis L.

In one aspect, at least a portion of the headband 30 is formed of elastic material 33. The elastic material can be formed of stretchable fabric, such as two or four-way stretch fabrics. As used herein, the term two-way stretch fabric is understood to have its widely accepted meaning of a fabric that stretches in one axial direction, while the term four-way stretch fabric refers to a fabric, such as Spandex™, that stretches in both a crosswise and lengthwise direction. Further, as used herein, the term elastic material or elastic fabric, refers to a fabric containing interwoven or knit strands of flexible material that have shape memory properties that allow the material to stretch and return to its original shape.

The mount assembly 40 includes a first sheet of material 42 that is positioned in opposition to a second sheet of material 44. Each of the first and second sheets of material defines an elliptically shaped central opening 45. As shown, the opposed first and second sheets of material are connected about a portion of the respective circumferential edges 46 of the first and second sheets and define an elliptically shaped central opening 50 in the mount assembly. Further, the opposed first and second sheets of material define a circumferentially extending pocket 48 between the respective inner surfaces of the first and second sheets, the circumferential edges 46 and the elliptically shaped central opening 45 of each sheet of material.

The mount assembly 30 can be coupled to a portion of an exterior and/or interior surface of the headband such that a major axis of the elliptically shaped central opening 50 is positioned, in a non-worn position, at an acute angle γ with respect to the longitudinal axis of the headband. In one aspect, the acute angle γ can range from between about 30 degrees to about 60 degrees, preferably between about 40 degrees to about 50 degrees, and more preferably about 45 degrees. Further, as it is contemplated that the mount assembly 30 be formed of a fabric material, the ranges noted above for the acute angle γ contemplate a variance of 10% due to the flexible nature of the underling fabric material.

The headband 30 has an upper edge 32 and a lower edge 34. In one aspect, a portion of the circumferential edges 46 of the first and second sheets can be positioned proximate an upper edge 32 of the headband. In a further aspect, it is contemplated that a focus F of the elliptically shaped central opening 50 can be positioned proximate a lower edge 34 of the headband. Optionally, the focus F of the elliptically shaped central opening 50 can be positioned between the longitudinal axis L of the headband 30 and the lower edge 34 of the headband.

The strap 60 is mounted to and extends outwardly from a bottom portion of the circumferential edges 46 of the first and second sheets 42, 44 of the mount assembly along an axis that can be generally coaxial to the major axis of the elliptically shaped central opening 50. Similar to the properties of the materials forming the headband, it is contemplated that at least a portion of the strap 60 is formed of elastic material 63 to allow for conformance of the strap about the patient's head. As further shown, it is contemplated that a distal end portion 62 of the strap can be configured to be selectively and releasably coupled to a portion of the headband when the eye patch is positioned in mounted position, such that the eye patch is in a normal bridging mounted relation over an eye socket of a wearer to give full protection to and cover the desired eye. In operation, the strap 60 is passed under the patient's ear and continues about at least a portion of the rear portion of the patient's skull such that the distal end portion of the strap can selectively coupled to a rear portion of the headband 30. Further, because a least a portion of the strap 60 is formed of an elastic material, the strap can be stretched and subsequently coupled to the headband to ensure that a desired degree of tension is placed onto the strap 60 and the coupled mount assembly 40 to assure the seated secured relationship of the mount assembly to the eye socket bone construction is achieve.

In some embodiments, the distal end 62 of the strap 60 is attached headband by a movable attachment means that allows the positioning of the distal end of the strap relative to the headband to be adjusted to suit different individuals and or desired degree of compression. An example of the movable attachment means includes a hook-and-loop fastening mechanism such as a VELCRO™ brand hook-and-loop fastening mechanism. In one example, the loop portions of the hook-and-loop fastening mechanism can be mounted to a portion of an exterior surface of a rear portion 39 of the headband 30 and the complementary hook portions of the hook-and-loop fastening mechanism can be mounted to the distal end 62 of the strap 60. Optionally, the hook portions of the hook-and-loop fastening mechanism can be mounted to a portion of an exterior surface of a rear portion 39 of the headband 30 and the complementary loop portions of the hook-and-loop fastening mechanism can be mounted to the distal end 62 of the strap 60. In another exemplary and non-limiting aspect, and as illustrated, to allow for the reversible use of the eye patch 10, it is contemplated that loop portions of the hook-and-loop fastening mechanism can be mounted, in opposition, to portions of both the interior and exterior surfaces of a rear portion 39 of the headband 30 and the complementary hook portions of the hook-and-loop fastening mechanism can be mounted, in opposition, to portions of both an inner and outer surfaces of the distal end 62 of the strap 60.

The eye patch further comprises an eye shield 20 that is configured to be selectably seated within the pocket 38 of the mount assembly 40. In this aspect, a portion of respective circumferential edges of the first and second sheets 42, 44 of the mount assembly 40 defines an access opening that allows for access thereto the circumferentially extending pocket 48. The access opening has an elongate length that is configured to allow passage of the eye shield into the circumferentially extending pocket. In one exemplary aspect, and not meant to be limiting, the elongate length of the access opening can have a length that is at least the length of the minor axis of the elliptically shaped central opening 50. Further, it is contemplated that the assess opening can be configured to be selectively opened or sealed by the patient to selectively seat or position the eye shield 20 in position within the circumferentially extending pocket 48.

In one aspect, the eye shield 20 has a generally ovoid shape and a peripheral edge 22. It is further contemplated that the generally ovoid shape can further include a generally teardrop shape. In a further aspect, the eye shield can be rigid and the peripheral edge 22 can be shaped to conform to the facial contours of the patient. Optionally, it is contemplated that at least a portion of the eye shield can be deformable to allow the peripheral edge 22 of the eye shield 20 to deform to the facial contours of the patient under application of a compressive force. It is also contemplated that the eye shield can be formed from a rigid, plastically deformable, and preferably light-weight material, such as aluminum or any suitable plastic material having the requisite rigidity and deformability characteristics.

Further, the eye shield 20 can have a dome shape in cross-section such that the inner surface of the eye shield extends upwardly and away from the peripheral edge 22 of the eye shield. Optionally, the eye shield can further define a plurality of apertures that permit at least a limited degree of sight in the covered eye and that permits air to circulate in the enclosed space defined between the inner surface of the eye shield 20 and the covered eye of the patient when the eye patch is secured to the patient's head in the mounted position.

It is also contemplated that the eye shield 20 can include an annular cover member 24 that is configured to be mounted about the periphery of the eye shield to overlie the peripheral edge 22 of the eye shield. The annular cover member 24 can preferably be formed of a fabric cover and elastic means for maintaining the fabric about the peripheral edge of the eye shield. In one exemplary, and non-limiting, example, the elastic means can comprise a length of elastic material sewn into at least a portion of the interior surface of the annular cover member 24.

It should be emphasized that the above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims which follow, they are used only in a generic and descriptive sense, and not for the purposes of limiting the described invention, nor the claims which follow.

What is claimed is:

1. An eye patch for use by a patient, comprising:
a headband having a longitudinal axis, wherein at least a portion of the headband is formed of elastic material;
a mount assembly having first sheet of material positioned in opposition to a second sheet of material, the first and second sheets being connected about a portion of the respective circumferential edges of the first and second sheets and define a circumferentially extending pocket between the respective inner surfaces of the first and second sheets; wherein the opposed first and second sheets define an elliptically shaped central opening in the mount assembly; wherein the mount assembly is coupled to a portion of an exterior surface of the headband such that a major axis of the elliptically shaped central opening is positioned at an acute angle β with respect to the longitudinal axis of the headband; and
a strap mounted to and extending outwardly from a portion of the circumferential edges of the first and second sheets of the mount assembly along an axis generally coaxial to the major axis of the elliptically shaped central opening; wherein at least a portion of the strap is formed of elastic material; wherein a distal end portion of the strap is configured to be selectively and releasably coupled to a portion of the headband.

2. The eye patch of claim 1, wherein the headband has an upper edge and a lower edge.

3. The eye patch of claim 2, wherein a portion of the circumferential edges of the first and second sheets is positioned proximate an upper edge of the headband.

4. The eye patch of claim 2, wherein a focus of the elliptically shaped central opening is positioned proximate a lower edge of the headband.

5. The eye patch of claim 2, wherein a focus of the elliptically shaped central opening is positioned between the longitudinal axis of the headband and the lower edge of the headband.

6. The eye patch of claim 1, wherein the headband is continuous.

7. The eye patch of claim 1, wherein the eye patch is reversible and can be configured to be worn on either the left or right eye of the patient.

8. The eye patch of claim 1, wherein the acute angle β ranges from about 30 degrees to about 60 degrees.

9. The eye patch of claim 1, further comprising an eye shield having a generally ovoid shape and a peripheral edge, wherein the eye shield is configured to be selectably seated within the pocket of the mount assembly.

10. The eye patch of claim 9, wherein the generally ovoid shape is a generally teardrop shape.

11. The eye patch of claim 9, wherein the eye shield is rigid and the peripheral edge is shaped to conform to the facial contours of the patient.

12. The eye patch of claim 9, wherein at least a portion of the eye shield is deformable to allow the peripheral edge of the eye shield to deform to the facial contours of the patient.

13. The eye patch of claim 9, further comprising an annular cover member configured to be mounted about the periphery of the eye shield and overlie the peripheral edge of the eye shield.

14. The eye patch of claim 9, wherein a portion of respective circumferential edges of the first and second sheets of the mount assembly defines an access opening thereto the circumferentially extending pocket, wherein the access opening has an elongate length that is configured to allow passage of the eye shield into the circumferentially extending pocket; and wherein the assess opening is configured to be selectively opened or sealed by the patient to selectively seat the eye shield therein the circumferentially extending pocket.

14. The eye patch of claim 9, wherein the elongate length of the access opening has a length that is at least the length of the minor axis of the elliptically shaped central opening.

15. A reversible eye patch configured to be worn on either the left or right eye of the patient, comprising:
a continuous headband having a longitudinal axis, wherein at least a portion of the headband is formed of elastic material;
a mount assembly having first sheet of material positioned in opposition to a second sheet of material, the first and second sheets being connected about a portion of the respective circumferential edges of the first and second sheets and define a circumferentially extending pocket between the respective inner surfaces of the first and second sheets; wherein the opposed first and second sheets define an elliptically shaped central opening in the mount assembly; wherein the mount assembly is coupled to a portion of an exterior surface of the headband such that a major axis of the elliptically shaped central opening is positioned at an acute angle β with respect to the longitudinal axis of the headband;
an eye shield having a generally ovoid shape and a peripheral edge, wherein the eye shield is configured to be selectably seated within the pocket of the mount assembly, wherein a portion of respective circumferential edges of the first and second sheets of the mount assembly defines an access opening thereto the circumferentially extending pocket, wherein the access opening has an elongate length that is configured to allow passage of the eye shield into the circumferentially extending pocket; and wherein the assess opening is configured to be selectively opened or sealed by the patient to selectively seat the eye shield therein the circumferentially extending pocket; and
a strap mounted to and extending outwardly from a portion of the circumferential edges of the first and second sheets of the mount assembly along an axis generally coaxial to the major axis of the elliptically shaped central opening; wherein at least a portion of the strap is formed of elastic material; wherein a distal end portion of the strap is configured to be selectively and releasably coupled to a portion of the headband.

16. The eye patch of claim 15, wherein the headband has an upper edge and a lower edge.

17. The reversible eye patch of claim 16, wherein a portion of the circumferential edges of the first and second sheets is positioned proximate an upper edge of the headband.

18. The reversible eye patch of claim 16 wherein a focus of the elliptically shaped central opening is positioned proximate a lower edge of the headband.

19. The reversible eye patch of claim 16, wherein a focus of the elliptically shaped central opening is positioned between the longitudinal axis of the headband and the lower edge of the headband.

20. The reversible eye patch of claim 15, wherein the acute angle β ranges from about 30 degrees to about 60 degrees.

21. The reversible eye patch of claim 15, wherein the eye shield is rigid and the peripheral edge is shaped to conform to the facial contours of the patient.

22. The reversible eye patch of claim 15, wherein at least a portion of the eye shield is deformable to allow the peripheral edge of the eye shield to deform to the facial contours of the patient.

23. The reversible eye patch of claim 15, further comprising an annular cover member configured to be mounted about the periphery of the eye shield and overlie the peripheral edge of the eye shield.

24. The reversible eye patch of claim 15, wherein the elongate length of the access opening has a length that is at least the length of the minor axis of the elliptically shaped central opening.
